SEQUENCE LISTING (1 sequence)

SEQ ID NO: 1 - protein, 333 aa, Artificial Sequence
  TACI-Fc fusion protein
SRVDQEERFPQGLWTGVAMRSCPEEQYWDPLLGTCMSCKTICNHQSQRTCAAFCRSLSCRKEQGKFYDHLLRDCISCASICGQHPKQCAYFKENKLRSPVNLPPELDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

(19) United States
(12) Patent Application Publication
(10) Pub. No.: US 2021/0087253 A1
(43) Pub. Date: Mar. 25, 2021

(54) APPLICATION OF TACI-FC FUSION PROTEIN IN PREPARATION OF DRUGS FOR TREATING NEUROMYELITIS OPTICA SPECTRUM DISORDERS AND MULTIPLE SCLEROSIS

(71) Applicant: REMEGEN, LTD., Shandong (CN)

(72) Inventors: Jianmin Fang, Shandong (CN); Jing Jiang, Shandong (CN); Shenjun Li, Shandong (CN); Min Huang, Shandong (CN)

(21) Appl. No.: 16/760,413

(22) PCT Filed: May 15, 2019
(86) PCT No.: PCT/CN2019/086990

§ 371 Date: Apr. 29, 2020

(30) Foreign Application Priority Data
May 25, 2018 (CN) 201810512508.8

(51) Int. Cl. C07K 14/705; A61P 25/00; A61P 37/02; A61K 9/00; A61K 38/17

(52) U.S. Cl. CPC C07K 14/70578; A61P 25/00; A61P 37/02; C07K 2319/30; A61K 9/0019; A61K 38/1793; A61K 9/0053

(57) ABSTRACT

The present invention relates to the field of B-lymphocyte stimulator receptor-antibody fusion proteins for treating autoimmune diseases, and in particular to a TACI-Fc fusion protein in preparation of drugs for treating neuromyelitis optica spectrum disorders (NMOSD) and multiple sclerosis (MS).

### FIELD OF THE INVENTION

The present invention relates to the field of using a fusion protein of B-lymphocyte stimulating factor receptor and antibody to treat autoimmune diseases, and more specifically, it relates to the use of TACI-Fc fusion protein for the manufacture of a medicament for the treatment of neuromyelitis optica spectrum disorders (NMOSD) and multiple sclerosis (MS).

### BACKGROUND OF THE INVENTION

Neuromyelitis optica (NMO) is an acute or subacute demyelinating disease in which the optic nerve and the spinal cord are affected simultaneously or sequentially. The disease was first described by Devic in 1894. It is clinically characterized by acute or subacute onset of blindness in one or both eyes. It is accompanied by transversal or ascending myelitis before or after a few days or weeks. Research data show that NMO accounts for up to 22% of all demyelinating diseases, a low proportion in Western countries and a high proportion in non-Caucasians. The disease is mainly characterized by B-cell immune disorders. Clinically, it is mainly characterized in that the optic nerve and spinal cord are affected simultaneously or sequentially. The lesions are mostly diffuse and can affect one or several spinal cord segments. The nature of the disease is demyelination of varying severity. Sclerotic plaques and necrotic voids formed with varying degrees of severity.

Epidemiological data show that the prevalence of neuromyelitis optica (NMO) is 0.3-4.4/100,000. The annual incidence is 0.05-0.4/100,000. Both men and women can develop the disease, and the female/male ratio is about 9-12:1. The average age of onset is 30-40 years, and about 10% of NMO patients are younger than 18 years. Familial NMO cases are rare, accounting for less than 3% of all diagnosed NMOs. Asian and Caucasian populations are associated with NMO susceptibility. It can be seen that genetic factors play a role in the pathogenesis of NMO, but they are not the main cause.

Neuromyelitis optica spectrum disorders (NMOSD) is a general term for a group of diseases. In 2014, the NMO Diagnosis International Expert Group (IPND) reached a new diagnostic standard for NMOSD using a systematic review and electronic questionnaire. NMO was integrated into NMOSD's standard system, and according to the status of AQP4 antibodies, it was divided into two categories: AQP antibody positive and AQP4 antibody negative. The main clinical types of the disease are: neuromyelitis optica (NMO), recurrent optic neuritis (RON), longitudinally extending transverse myelitis (LETM), optic-spinal form of multiple sclerosis (OSMS), long-term transverse myelitis, Unilateral or bilateral optic neuritis, optic neuritis or myelitis accompanying with autoimmune diseases, optic neuritis or myelitis accompanying with symptomatic or asymptomatic brain lesions, etc. NMOSD is different between the East and West populations. NMOSD is common in China, the patient population is large, and the number of patients is increasing year by year.

Multiple sclerosis (MS) is also an autoimmune disease with white matter demyelination of the central nervous system as the main pathological feature. It is mainly characterized by T cell immune disorders. Among them, multiple demyelinating plaques in the white matter of the central nervous system is a characteristic pathological change that occurs frequently in the white matter around the lateral ventricle, optic nerve, spinal cord, cerebellum, and brainstem. The disease is more common in early adulthood, with more women than men, and most patients present with recurrent neurological dysfunction, with multiple remissions and relapses, the condition is worsened. Clinical studies have confirmed that patients with multiple sclerosis have limb numbness, pain, or paresthesia most, followed by muscle weakness.

The characteristic pathological changes of multiple sclerosis are multiple demyelinating plaques in the white matter of the central nervous system, mostly located around the lateral ventricle, with reactive glial hyperplasia, and may also have axonal damage. Lesions can affect the white matter of the brain, spinal cord, brain stem, cerebellum, and optic nerve. Cerebral and spinal coronal sections can be seen by the naked eye with more pink-gray scattered demyelinating lesions of different shapes, varying in size, 1-20 mm in diameter, with semi-oval centers and around the ventricles, especially the lateral ventricle anterior horns. Microscopy showed that the myelin sheath disintegrated and lost during the acute phase, the axons were relatively intact, and the oligodendrocytes were slightly degenerated and proliferated. Infiltration of inflammatory cells (monocytes, lymph and plasma cells) around the small veins was seen. In the late stage of the disease, the axons were disintegrated, and the number of nerve cells was reduced, replaced by sclerotic plaques formed by glial cells.

According to the incidence, MS is divided into primary MS (PPMS), recurrent MS (RRMS) and secondary MS (SPMS). Symptoms of primary MS continue to worsen, but there is usually no significant relapse or remission period. Approximately 15% of patients with MS are diagnosed with this type, and the remaining 85% are MS in relapsed form (RMS, including RRMS). And SPMS), patients with relapsed MS usually experience a cycle of worsening, recovery, and further worsening. This is because the inflammation subsides and our body initiates the repair mechanism. However, the immune system will continue to attack the myelin sheath after repair, causing the patient's condition to recur. Once inflammation recurs in the same location, secondary MS occurs, and 80% of patients with recurrent MS eventually develop secondary MS.

There are not many drugs that can effectively treat neuromyelitis optica spectrum disorders (NMOSD) and multiple sclerosis (MS).

For NMOSD treatment, the FDA has granted the anti-CD19 monoclonal antibody MEDI-551 the orphan drug status for the treatment of neuromyelitis optica (NMO) and neuromyelitis optica spectrum disorders (NMOSD). MEDI-551 is a humanized monoclonal antibody that targets and binds to the CD19 protein with high affinity. The CD19 protein is expressed on a wide range of B cells, including specific B cells called plasmablasts. Studies have shown that the autoantibody AQP4-Ab (or NMO-IgG) against AQP4 produced in these plasmablasts plays a key role in the pathogenesis of NMO. MEDI-551 can directly bind to the CD19 protein on the surface of plasmablasts and deplete these plasmablasts. MEDI-551 is currently in a global clinical trial phase and is being investigated for potential treatments for NMO and NMOSD. In addition, the treatment of diffuse large B-cell lymphoma with MEDI-551 is already in a phase II clinical trial. Prior to this, no drugs have been approved for the treatment of NMO and NMOSD.

For MS treatment, as there is no method of cure, early treatment is advocated: the treatment in the acute phase is mainly to reduce symptoms and improve the degree of disability as soon as possible; the treatment in the remission phase (that is, a disease modifying treatment (DMT)) is mainly to reduce the relapse rate and reduce brain tissue and spinal cord lesions, delay the disease process, and improve the quality of life of patients. The treatment thereof can be divided into acute phase treatment and remission phase treatment.

For acute phase treatment, intravenous methylprednisolone (IVMP) is preferred: the dose is 1 g intravenous infusion (1 time/d), the dose is halved every 3 days until the drug is discontinued, and it is taken orally when it is reduced to 64 mg. Short-term hormone shock treatment can shorten the recovery time of neurological damage in the acute phase, but the long-term efficacy is not yet certain. Regular hormone shock therapy may improve the long-term prognosis of patients with relapsing-remitting multiple sclerosis. If the effect of hormone therapy is unsatisfactory or the patients cannot tolerate its adverse reactions, large doses of intravenous immunoglobulin and/or plasma exchange therapy can be used.

For remission phase treatment, the US Food and Drug Administration (FDA) has approved 10 disease-modifying drugs (DMDs) for multiple sclerosis. Five of these are first-line drugs, including glatiramer acetate, fingolimod (FTY-720), and others. Five second-line drugs include natalizumab, mitoxantrone, terifunomide, dimethyl fumarate (DMF), and cladribine. The above drugs can effectively reduce the progress of relapse and disability, and improve the quality of life of patients. Therefore, it is very necessary to perform immunomodulatory treatment for relapsing-remitting multiple sclerosis. At present, there are no domestic original research related drugs on the market.

TACI (Transmembrane Activator and CAML Interactor) was first discovered by scientists at the famous American academic institution St. Jude Children's Hospital, VonBulow and Bram, but later studies found that the natural sequence of the extracellular region of TACI has the problem of easy degradation of the protein and is not suitable for production as a drug. Since then, several companies have made improvements to TACI's original molecule, including Genentech, Amgen, and ZymoGenetics, which are well-known biotech companies in the United States. At present, the TACI fusion protein Atacicept produced by ZymoGenetics combined with Merck Serono has conducted clinical trials for diseases such as systemic lupus erythematosus (SLE), RA, and lymphoma. The results show that Atacicept has a clear biological activity without obvious side effects.

However, the latest data show that Atacicept's clinical trial (Feb. 17, 2016) against optic neuritis, a disease of the neuromyelitis optic a spectrum disorders NMOSD, was failed, suggesting that TACI fusion protein may not be used to treat NMOSD. Two years ago, a randomized, double-blind, placebo-controlled phase II clinical trial of Atacicept in the treatment of MS was also failed (Mar. 20, 2014). The result showed that atacicept treatment will lead to an increase in MS recurrence rate. Therefore, prior to the present invention, data in the prior art indicate that a TACI fusion protein may not be useful in the treatment of MS.

The inventor has disclosed a new TACI-Fc fusion protein in multiple patent documents, such as CN101323643B, CN102085367B, and CN102085368B, the entire contents of which are incorporated herein by reference. Contrary to the clinical results of Atacicept, it was unexpectedly found in further studies that the above-mentioned TACI-Fc fusion protein can effectively treat NMOSD and MS.

### SUMMARY OF THE INVENTION

One object of the present invention is to provide a use of TACI-Fc fusion protein for the preparation of a drug for treating neuromyelitis optica spectrum disorders (NMOSD).

In one aspect, the present invention relates to the use of TACI-Fc fusion protein with a sequence represented by SEQ ID: 1 for the preparation of a drug for treating neuromyelitis optica (NMO).

In another aspect, the present invention relates to the use of TACI-Fc fusion protein with a sequence represented by SEQ ID: 1 for the preparation of a drug for treating neuromyelitis optica spectrum disorders (NMOSD).

In another aspect, the present invention relates to the use of TACI-Fc fusion protein with a sequence represented by SEQ ID: 1 for the preparation of a drug for treating multiple sclerosis (MS).

In another aspect, the neuromyelitis optica spectrum disorders (NMOSD) include: neuromyelitis optica, recurrent optic neuritis, longitudinally extending transverse myelitis, optic nerve spinal multiple sclerosis, long-term transverse myelitis, unilateral or bilateral optic neuritis, optic neuritis or myelitis accompanying with autoimmune disease, optic neuritis or myelitis accompanying with symptomatic or asymptomatic intracerebral lesions.

In another aspect, the present application relates to a method of treating neuromyelitis optica spectrum disorders (NMOSD) or neuromyelitis optica (NMO), which comprises administering to a patient a therapeutically effective amount of TACI-Fc fusion protein having a sequence represented by SEQ ID NO: 1.

In another aspect, the present invention relates to TACI-Fc fusion protein having a sequence represented by SEQ ID NO: 1, for use in treating neuromyelitis optica (NMO) or neuromyelitis optica spectrum disorders (NMOSD).

In particular, the TACI portion of the TACI-Fc fusion protein comprises: the amino terminal region sequence starting from amino acid position 13 in the extracellular region of TACI, the entire cysteine-rich region, and a partial sequence of the stalk region. The immunoglobulin Fc portion of the fusion protein comprises: a hinge region, CH2 region and CH3 region. The TACI sequence and the Fc sequence are linked directly or via a linker sequence.

In some embodiments, the TACI sequence of the TACI-Fc fusion protein is preferably positions 13-108 or 13-118 of the amino acid sequence of TACI.

In some embodiments, the immunoglobulin Fc sequence is selected from the Fc of human or animal immunoglobulin, which is a full-length or partial Fc sequence. Fc is selected from IgG, IgM, IgD and IgA, including various subtypes, such as IgG1, IgG2, IgG3, IgG4, and preferably IgG1.

In some embodiments, the TACI sequence of the TACI-Fc fusion protein and the immunoglobulin Fc sequence can be linked directly or via a linker sequence. If fusion is performed via a linker sequence, fusion via the linker sequence 9Gly is preferred.

In a preferred embodiment, the TACI-Fc fusion protein of the present invention is preferably formed by positions 13-118 amino acids of TACI and Fc of immunoglobulin IgG1.

The sequence is shown as follow.

SEQ ID NO: 1
SerArgValAspGl [symbol] Gl [symbol] Gl [symbol] ArgPheProGl [symbol] GlyLeuTrpThrGlyValAlaMetArg
1 5 10 15 20
SerCysProGl [symbol] Gl [symbol] Gl [symbol] TyrTrpAspProLeuLeuGlyThrCysMetSerCysLysThr
21 25 30 35 40
IleCysAsnHisGl [symbol] SerGl [symbol] ArgThrCysAlaAlaPheCysArgSerLeuSerCysArg
41 45 50 55 60
LysGl [symbol] Gl [symbol] GlyLysPheThrAspHisLeuLeuArgAspCysIleSerCysAlaSerIle
61 65 70 75 80
CysGlyGl [symbol] HisProLysGl [symbol] CysAlaTyrPheCysGl [symbol] AsnLysLeuArgSerProVal
81 85 90 95 100
AsnLeuProProGl [symbol] LeuAspLysProHisThyCysProLeuCysProAlaProGl [symbol] Leu
101 105 110 115 120
LeuGlyGlyProSerValPheLeuPheProProLysProLysAspThrLeuMetIleSer
121 125 130 135 140
ArgThrProGl [symbol] ValThrCysValValValAspSerValHisGl [symbol] AspProGl [symbol] ValLys
141 145 150 155 160
PheAsnTrpTyrValAspGlyValGl [symbol] ValHisAsnAlaLysThrLysProArgGl [symbol] Gl [symbol]
161 165 170 175 180
Gl [symbol] TyrAsnSerThrTyrArgValValSerValLeuThrValLeuHisGl [symbol] AspTrpLeu
181 185 190 195 200
AsnGlyLysGl [symbol] TyrLysCysLysValSerAsnLysAlaLeuProAlaProIleGl [symbol] Lys
201 205 210 215 220
ThrIleSerLysAlaLysGlyGl [symbol] ProArgGl [symbol] ProGl [symbol] ValTyrThrLeuProProSer
221 225 230 235 240
ArgAspGl [symbol] LeuThrLysAsnGl [symbol] ValSerLeuThrCysLeuValLysGlyPheTyrPro
241 245 250 255 260
SerAspIleAlaValGl [symbol] TrpGl [symbol] SerAsnGlyGl [symbol] ProGl [symbol] AsnAsnTyrLysAlaThr
261 265 270 275 280
ProProValLeuAspSerAspGlySerPhePheLeuTyrSerLysLeuThrValAspLys
281 285 290 295 300
SerArgTrpGl [symbol] Gl [symbol] GlyAsnValPheSerCysSerValMetHisGl [symbol] AlaLeuHisAsn
301 305 310 315 320
HisTyrThrGl [symbol] LysSerLeuSerLeuSerProGlyLys
321 325 330 333

[symbol]

indicates data missing or illegible when filed

In a specific embodiment, the present invention relates to TACI-Fc fusion protein having the amino acid sequence represented by SEQ ID NO: 1, namely RCT18.

The TACI-Fc fusion protein of the present invention can be used with a pharmaceutically acceptable carrier to prepare drugs for treating neuromyelitis optica spectrum disorders (NMOSD) or multiple sclerosis (MS) by conventional methods in the art. The carrier includes an excipient, a diluent, a filler, an adhesive, a wetting agent, a disintegrating agent, an absorption enhancer, a surfactant, an adsorption carrier, a stabilizer, and the like.

The drugs of the present invention can be made into various clinically acceptable dosage forms, including but not limited to oral preparations or injection preparations, preferably injections.

In certain embodiments, the TACI-Fc fusion protein of the invention is administered to the patient once every at least 28 days, every at least 27 days, every at least 26 days, every at least 25 days, every at least 24 days, every at least 23 days, every at least 22 days, every at least 21 days, every at least 20 days, every at least 19 days, every at least 18 days, every at least 17 days, every at least 16 days, every at least 15 days, every at least 14 days, every at least 13 days, every at least 12 days, every at least 11 days, every at least 10 days, every at least 9 days, every at least 8 days, every at least 7 days, every at least 6 days, every at least 5 days, every at least 4 days, every at least 3 days, every at least 2 days or every at least 1 day.

In certain embodiments, the TACI-Fc fusion protein of the invention is administered to the patient once every at most 28 days, every at most 21 days, every at most 15 days, every at most 14 days, every at most 13 days, every at most 12 days, every at most 11 days, every at most 10 days, every at most 9 days, every at most 8 days, every at most 7 days, every at most 6 days, every at most 5 days, every at most 4 days, every at most 3 days, every at most 2 days or every at most 1 day.

In certain embodiments, the TACI-Fc fusion protein of the invention is administered to the patient once every 6-8 days, every 13-15 days, every 20-22 days, or every 27-29 days.

In certain embodiments, the TACI-Fc fusion protein of the invention is administered to the patient once weekly, every two weeks, every three weeks, every four weeks, every five weeks, every six weeks, or every seven weeks.

In certain embodiments, the TACI-Fc fusion protein of the present invention is administered at a dose of 25 mg/time, 40 mg/time, 75 mg/time, 80 mg/time, 120 mg/time, 150 mg/time, 160 mg/time, 200 mg/time, 240 mg/time 280 mg/time, 320 mg/time, 360 mg/time, 400 mg/time, 440 mg/time, 480 mg/time, 520 mg/time, 560 mg/time, or 600 mg/time.

In certain embodiments, the subjects meet the international consensus diagnostic criteria for neuromyelitis optic a spectrum disorders (NMOSD) in 2015, and/or is AQP4-IgG positive.

The TACI-Fc fusion protein of the present invention can be administered by any suitable route, such as intranasal, intradermal, subcutaneous, intramuscular, or intravenous administration.

As used herein, “a therapeutically effective amount” or “an effective amount” refers to a dose sufficient to show its benefit to the patient being administered. The actual amount administered, as well as the rate and time course of administration, will depend on the condition and severity of the person being treated. The prescription of treatment (e.g., decisions on dosage, etc.) is ultimately the responsibility and reliance of general practitioners and other physicians to make decisions, usually considering the disease being treated, the condition of the individual patient, the location of delivery, the method of administration, and other factors known for doctors.

As used herein, “TACI-Fc fusion protein”, “TACI-Fc”, and “TACI-Ig” can be used interchangeably, and all represent a fusion protein of TACI and the Fc region of an immunoglobulin.

In the present invention, by establishing an EAE mouse model and scoring the clinical symptoms of the animal, the therapeutic effect of the TACI-Fc on the EAE model mouse is analyzed at a pharmacological level. and is confirmed by Experiments demonstrated that, the fusion protein of the present invention would not affect the apoptosis and proliferation of normal peripheral lymphocytes. The fusion protein can reduce the secretion of peripheral cytokines such as APPIL and inhibit the proliferation of activated B cells, thereby reducing the inflammation infiltration of CNS, improving the degree of demyelination of the spinal cord and other parts and exerting a treatment effect. It has a significant treatment effect in the EAE mouse model.

In addition, the TACI-Fc fusion protein of the present invention can retain most of the amino acid residues in the amino terminal region of TACI, which greatly improves the affinity between the fusion protein and Blys while compared with the prior art, giving an increase of the affinity between the drug and the target. It means that the use concentration of the drug for achieving the expected efficacy is reduced, and the side effects of drug may be reduced, the cost for patients is reduced, and it is more suitable for general use and industrial production.

The applicant found a natural TACI degradation site through analysis of protein precursor processing enzyme artificial neural network. By retaining most of the amino acid residues in the amino terminal of TACI, the TACI-Fc fusion protein unexpectedly avoids the problem of TACI degradation, and presents better biological activity.

The applicant has unexpectedly found that the TACI-Fc fusion protein of the present invention can be used to treat neuromyelitis optica spectrum disorders (NMOSD) and multiple sclerosis (MS). The drug provided by the present invention for the treatment of neuromyelitis optica spectrum disorders (NMOSD) and multiple sclerosis (MS) has the advantages of high biological activity, small use amount, and good safety.

### BRIEF DESCRIPTION OF THE FIGURE

FIG. 1. Panel A shows the effect of RCT-18 on the proliferation of normal T cells in mice (by cell apoptosis assay); Panel B shows the relationship between RCT-18 concentration and the proliferation of normal T cells in mice.

FIG. 2. Panel A shows the effect of RCT-18 on the proliferation of normal B cells in mice (by cell cycle assay); Panel B shows the relationship between RCT-18 concentration and the proliferation of G1-stage normal B cells in mice.

FIG. 3 shows the clinical scoring of the treatment on EAE mice.

FIG. 4. Panel A shows the CNS inflammation infiltration status (LFB staining) after the TACI-Ig treatment; Panel B shows the CNS inflammation infiltration status (inflammatory cell counting) after the TACI-Ig treatment.

FIG. 5. Panel A shows the assessment of CNS demyelination (LFB staining) after the TACI-Ig treatment; Panel B shows the assessment of CNS demyelination (demyelination scoring) after the TACI-Ig treatment.

FIG. 6 shows the effect of TACI-Ig on the proliferation of B cells in the peripheral immune system.

FIG. 7 shows the effect of TACI-Ig on the secretion of peripheral APRIL cytokines.

### DETAILED DESCRIPTION OF THE INVENTION

The inventor analyzed the amino acid sequence of TACI protein through the artificial neural network system of protein precursor processing enzymes and found that the sequence of TACI extracellular region contains two PC digestion sites (the 9^{th} and the 135^{th} amino acids) and the other two sites whose scores are also close to the critical value of the PC digestion sites (the 12^{th} and the 120^{th} amino acids). Therefore, by avoiding these sites the inventor designed a TACI-Fc fusion protein. The obtained TACI-Fc fusion protein characterizes by comprising most of the sequence of the amino terminal region of TACI, the entire sequence of a cysteine-rich region, and a partial sequence of the stalk region.

The following examples are further illustrations and explanations of the present invention, and should not be considered as limiting the present invention.

### EXAMPLE 1: EFFECT OF TACI-FC FUSION PROTEIN ON THE PROLIFERATION AND APOPTOSIS OF LYMPHOCYTES

T cells and B cells were isolated from the spleen of normal mice by magnetic beads.

The spleen of C57/BL6 mice aged 6-8 weeks was isolated aseptically, placed on a 70 μm strainer in pre-cooled PBS, and then carefully grinded clockwise. The filtrate was put into a 15 ml centrifuge tube, and the red cells were broken. Blocking was performed by adding 50 μl/ml of rat serum, and the mixture was transferred to a 5ml round-bottomed tube. 50 μl/ml Isolation Cocktail was added to the tube and incubation was performed at room temperature for 10 min. The mixture was vortexed for 30 s, and then 75 μl/ml of beads were added and incubated at room temperature for 2.5 min. The final volume in the tube was adjusted to 2.5 ml. The tube was put in a magnet and stand at room temperature for 3 min The isolated T cells or B cells were separated and obtain as a suspension.

The RCT-18 dry powder (Rongchang Biopharmaceutical (Yantai) Co., Ltd.) was formulated into a concentration of 0.04 mg/ml, 0.4 mg/ml, and 0.8 mg/ml using water for injection. The volume for drug administration was calculated based on the body weight and administration dose of the experimental animals in different dose groups. Positive control: FTY-720 (2-amino-2-[2-(4-n-octylphenyl) ethyl]-1,3-propanediol hydrochloride), which was formulated with 2% ethanol into a concentration of 0.72 mg/ml. The volume for drug administration was calculated based on the body weight of the animals in the positive control group.

Cell apoptosis and proliferation were detected after adding different concentrations of TACI-Ig

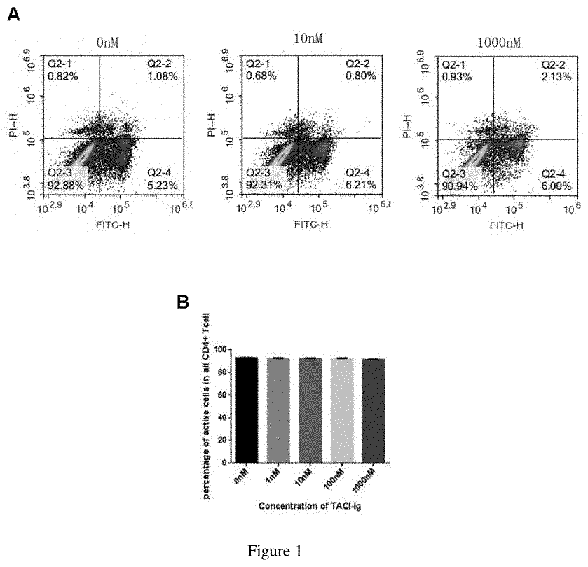

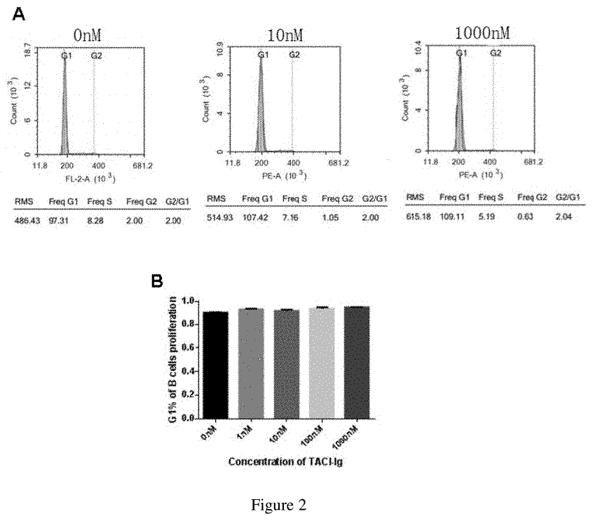

Primary cultured CD4+ T cells and CD19+ B cells were cultured in vitro for 24 hours, then 0 nM, 1 nM, 10 nM, 100 nM, 1000 nM of TACI-Ig were added for co-culture. The cells were collected 48 hours later and detected by using the FITC-Annexin apoptosis detection kit and the cell cycle detection kit according to the instructions. The experimental results were shown in FIG. 1 and FIG. 2. The results showed that different concentrations of TACI-Ig had no effect on the proliferation and apoptosis of normal T cells and B cells.

### EXAMPLE 2: ESTABLISHMENT OF THE EAE MOUSE MODEL

C57BL/6J female mice, aged 8-10 weeks, were each injected subcutaneously at three points 200 μl of complete Freund's adjuvant containing 100 μg MOG35-55 (GL Biochem) (containing heat-lethal Mycobacterium tuberculosis (MTB)) (H37Ra strain; Difco) in their buttocks. The specific steps were as follows: (1) MOG33-35 was dissolved in PBS and formulated into solution A with an initial concentration of 1 mg/ml; (2) a certain amount of MTB was weighed and put into a mortar, and IFA (Incomplete Freund's adjuvant; Sigma-Aldrich) was slowly added into the mortar with the same volume as PBS; MTB was grinded during adding to prepare 5 mg/ml of solution B; (3) two glass syringes were used to aspirate solution A and B respectively and connected with a tee, mixing was performed by pushing back and forth on the ice until pushing became difficult. Then a drop of the suspension was added to water. If diffusion was not present, the suspension was used for injection to establish the model. The final concentration of MOG was 0.5 mg/ml, and the final concentration of MTB was 2.5 mg/ml. All operations were performed on ice. On the day of modeling, each mouse was injected intraperitoneally with 200 μl of PBS containing 200 ng of PTX (pertussis toxin; Difco laboratories), and reinjected at the same dose 48 hours later.

After the administration, the animals entered the onset period after about 10 days of the latent period, and were scored according to the clinical symptoms. Level 0: no clinical symptoms; Level 1: limp tail; Level 2: limp tail and weakness of hind legs; Level 3: mild paralysis of the legs; Level 4: severe paralysis of the legs, unable to recover after passive rollover; Level 5: on the verge of death, or death.

### EXAMPLE 3 THE EAE MOUSE TREATMENT PROTOCOL

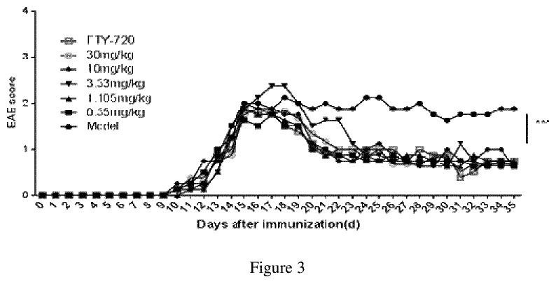

Animals were divided into negative control group, positive control group, and experimental group. The experimental group was given an administration dose of 0.350, 1.105, 3.333, 10, and 30 mg/kg. The positive control group was given FTY-720 at a dose of 5 mg/kg. Intraperitoneal injection was performed every other day from the peak of the onset period (i.e., the 16^{th} day after immunization), and the treatment was continued until the inflammation resolution phase (i.e., the 35^{th} day). The negative control group was injected with saline at the same time point. The scores and weights of the mice were recorded daily, and the scores were evaluated blindly by an independent person. According to the experimental results shown in FIG. 3, after the TACI-Ig treatment, the clinical symptoms of the EAE mice were significantly ameliorated, the clinical score was statistically different from the negative control, and there was no significant difference from the positive control group. In addition, ELISA detection showed that the APRIL levels of each group were significantly reduced after treatment (P<0.01).

### EXAMPLE 4 HISTOMORPHOLOGICAL DETECTION

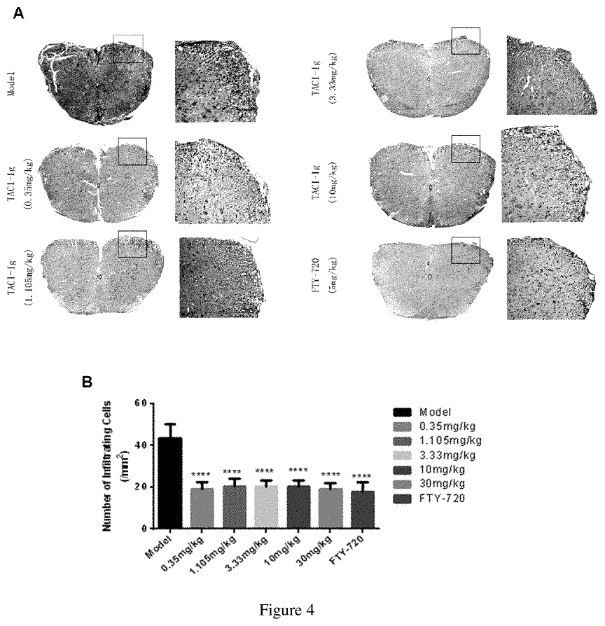

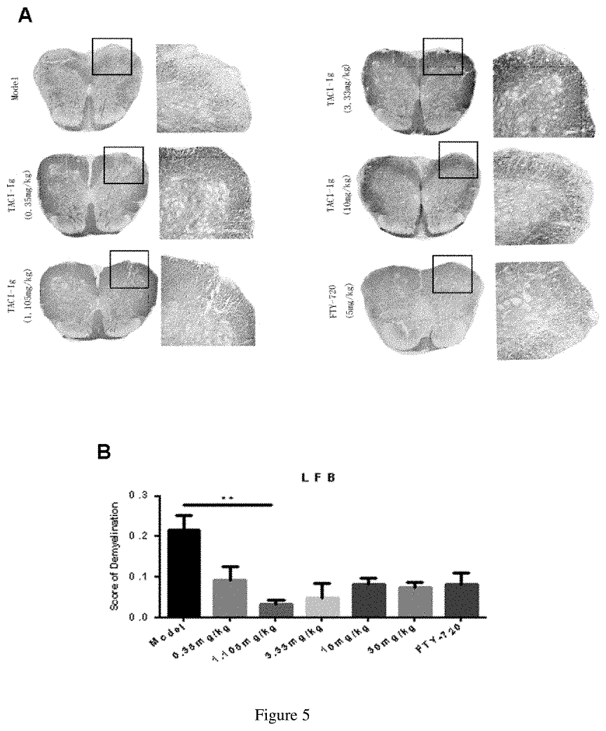

The mice were sacrificed on the 35^{th} day after immunization. The spinal cord lumbar swelling segments were taken and immersed in 4% paraformaldehyde for fixing. The sample was then embedded in paraffin and cut into slices with a thickness of 5 μm. After dewaxing and gradient hydration, HE staining and LFB staining were performed. After staining, observation was performed under an inverted microscope, and images were captured under 4× and 20× photos. For HE staining, inflammatory cells were counted from 10 spinal cord white matter areas with an area of 1 mm² selected randomly by an independent person, and the number of inflammatory cells per square millimeter was calculated for statistical analysis. For LFB, IPP6.0 software was used to choose all demyelinated regions and the total white matter region of the spinal cord white matter region, and the area proportion of the demyelinated regions to the total white matter region was calculated. The experimental results were shown in FIG. 4 and FIG. 5. The results showed that the degrees of demyelination of each treatment group were significantly ameliorated. It indicated that TACI-Ig of the present invention can effectively treat or alleviate NMOSD and MS.

### EXAMPLE 5 CHANGES IN PERIPHERAL IMMUNE LEVELS

After sacrifice, the spleen was taken out and prepared into a single-cell suspension. The cell suspension was divided into two groups, one group was stimulated by PTX-pertussis toxin, fixed and permeated, and then incubated with CD4, IL-4, IL-17A and IFN-γ antibodies; another group was incubated with CD19, CD21/35, CD23, IgM, IgD surface antibodies. After incubation, the proportion of each type of cell was detected by flow cytometry.

Peripheral blood sample was collected and allowed to stand for 2 hours. After centrifugation at 2300×g for 10 min, the supernatant was collected and subjected to ELISA for APRIL and BLyS according to the instructions of the corresponding kits.

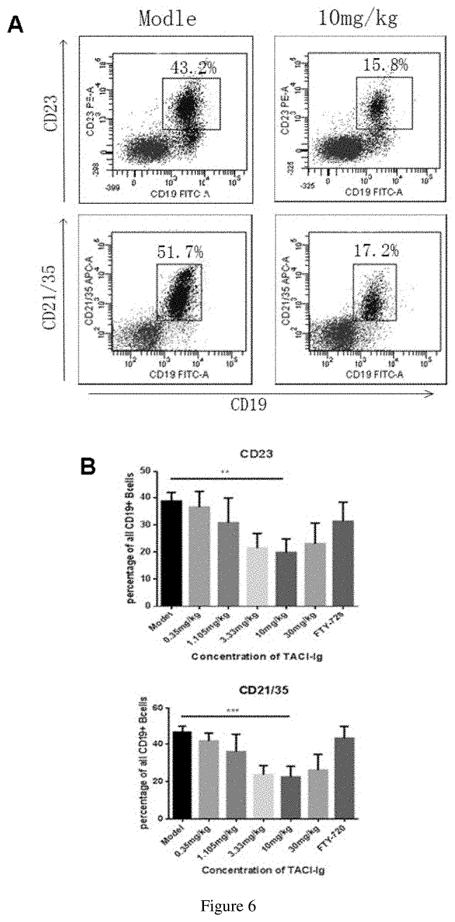

According to the experimental result shown in FIG. 6, after treatment, the T cell subsets such as Th1, Th2, Th17, and memory B cells (CD19CD27) in each group had no significant changes. CD21/35 and CD23 are surface markers of activated B cells. After treatment, activated B cells in the 10 mg/kg treatment group decreased significantly (P<0.01), and there was no significant difference in the 0.35 mg/kg group (P>0.01). This result indicated that the TACI-Ig of the present invention may achieve effective treatment or remission of NMOSD and MS by inhibiting B cell activation.

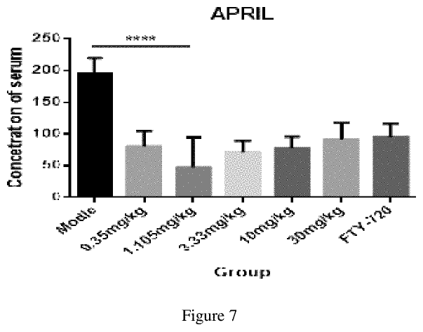

In addition, the experimental result in FIG. 7 showed that the APRIL level of each treatment group was significantly decreased after treatment (P<0.01).

The data in the experiments of the present application was shown as averages (S.D.). One-way ANOVA statistical analysis was performed by GraphPad Prism 6.0, and P<0.05 was set as the statistical level.

### Overall Conclusion

TACI-Ig had no significant effect on the proliferation and apoptosis of normal T cells and B cells in vitro. It had a significant therapeutic effect on the EAE mice. After treatment, the degree of CNS demyelination and inflammation infiltration in mice was significantly reduced and ameliorated, proving that the TACI-Fc fusion protein of the present invention can effectively treat NMOSD and MS.

### EXAMPLE 6 CLINICAL TRIAL OF RCT-18 FUSION PROTEIN

### 1. Clinical Trial Protocol Design

This clinical trial protocol used a sample grouping model of parallel randomized grouping and a double-blind test verification method was adopted to conduct a clinical trial for the treatment effect of neuromyelitis optica spectrum disorders (NMOSD).

### 2. Subject Selection Criteria

1) patients who met the international consensus diagnostic criteria for neuromyelitis optica spectrum disorders (NMOSD) in 2015 and were AQP4-IgG positive (according to the test results within 24 weeks at the time of signing the informed consent);

2) male or female, aged 18 to 65;

3) EDSS score 7.5;

4) female subjects of childbearing potential were subjected to pregnancy test during the screening period, and the result should be negative; and effective contraceptives should be used during the study;

5) experienced at least 2 relapses within 2 years prior to randomization, and/or experienced at least 1 relapse within 1 year prior to randomization;

6) voluntarily signed the informed consent.

### 3. Subject Exclusion Criteria

1) laboratory indicators that the subject was needed to be excluded include, but were not limited to, the following indicators: white blood cell count<3×10⁹/L, neutrophil<1.5×10⁹/L, hemoglobin<85 g/L, platelet count<80×10⁹/L, serum creatinine>1.5×ULN, total bilirubin>1.5×ULN, AST (GOT)>3×ULN, ALT (GPT)>3×ULN, alkaline phosphatase>2×ULN. AST, aspartate aminotransferase; GOT, glutamic oxalacetic transaminase; ALT, alanine aminotransferase; GPT, glutamic-pyruvic transaminase;

2) subjects who suffered from active hepatitis or a severe liver disease or had a history of that. Based on the following HBsAg, anti-HBc and anti-HBs antibody detection results, there was serological evidence of hepatitis B virus infection: HBsAg-positive patients should be excluded; HBsAg-negative but anti-HBc antibody-positive patients, regardless of whether the anti-HBs antibody was positive or negative, all needed to detect HBV-DNA to determine his/her condition: if HBV-DNA was positive, the patient needed to be excluded; if HBV-DNA was negative, the patient can participate in the trial;

3) in addition to neuromyelitis optica spectrum disorders, patients with other chronic active immune system diseases or with stable conditions but required glucocorticoid therapy were excluded, such as rheumatoid arthritis, scleroderma, Sjgren's Syndrome, ulcerative colitis, AIDS, genetic immunodeficiencies or drug-induced immunodeficiencies; patients with only positive autoantibodies but no clinical manifestations can be included into the groups for the trial; patients who used glucocorticoid maintenance therapy before randomization can participate in the trial after stopping using the drug;

4) pregnant women, lactating women, and patients with birth plan during the trial;

5) allergic reactions: patients with a history of allergies to parenteral contrast agents, human biological products;

6) patients who had received a live vaccine within 28 days prior to randomization, except those who had received herpes zoster vaccine;

7) patients who had used rituximab or other monoclonal antibodies within 6 months before randomization;

8) patients who used immunoglobulin (IVIG) for intravenous injection within 28 days before randomization;

9) patients who had received hematopoietic stem cell transplantation and lymphatic irradiation before randomization;

10) patients who took Azathioprine (AZA, half-life t_{1/2}=6 hrs), Mycophenolate Mofetil (t_{1/2}=16 hrs), Leflunomide (LEF, t_{1/2}=14.7 hrs), Tacrolimus (t_{1/2}=43 hrs), Teriflunomide (t_{1/2}=18 days), Cyclosporin (CsA, t_{1/2}=27 hrs.), Methotrexate (MTX, t_{1/2}=14 hrs), Mitoxantrone (NVT, t_{1/2}=37 hrs), Cyclophosphamide (CTX, t_{1/2}=6 hrs) and other immunosuppressive agents before randomization. Patients can be enrolled if the discontinuation interval was more than 5 times the half-life in addition to leflunomide and teriflunomide. For Leflunomide and teriflunomide, cholestyramine was needed to be taken for elution. The drugs can be discontinued and the following measures can be taken: taking 8 grams of cholestyramine 3 times a day for 11 days, if the dose of 8g cannot be tolerated, then changing the dose to 4 grams per time orally at the same time and frequency;

11) patients who had been given any clinical trial drug within 28 days before randomization or within 5 times the half-life of the trial drug (whichever was shorter);

12) patients with symptoms of severe mental illness and clinical incompatibility;

13) patients with malignant tumors;

14) patients who experienced any of the following events during the 12 weeks prior to randomization: myocardial infarction, unstable ischemic heart disease, stroke, or New York Heart Association Grade IV heart failure;

15) subjects who infected shingles, were HCV antibody positive or HIV antibody positive during the screening period;

16) patients who were unable to undergo magnetic resonance imaging detection during the trial; and

17) patients who the investigator considered as unsuitable for the trial.

### 4 Administration Plan

1) treatment group: recombinant human B lymphocyte stimulating factor receptor-antibody fusion protein lyophilized powder (specification: 80 mg/branch) for injection, subcutaneous injection, once a week.

2) control group: placebo lyophilized powder (specification: 80 mg/branch), subcutaneous injection, once a week.

1. A method for treating a neuromyelitis optica spectrum disorder (NMOSD) comprising administering TACI-Fc fusion protein having a sequence represented by SEQ ID NO: 1 to a subject in need thereof.

2. The method according to claim 1, wherein the neuromyelitis optica spectrum disorder (NMOSD) is selected from the group consisting of neuromyelitis optica, recurrent optic neuritis, longitudinally extending transverse myelitis, optic-spinal form of multiple sclerosis, long-term transverse myelitis, unilateral optic neuritis, bilateral optic neuritis, optic neuritis, myelitis accompanying autoimmune disease, myelitis accompanying symptomatic, and asymptomatic intracranial lesions.

3. The method according to claim 2, wherein the neuromyelitis optica spectrum disorder is neuromyelitis optica (NMO).

4. A method for treating multiple sclerosis (MS) comprising administering TACI-Fc fusion protein having a sequence represented by SEQ ID NO: 1 to a subject in need thereof.

5. The method according to claim 1, wherein the TACI-Fc fusion protein is administered in a pharmaceutically acceptable carrier.

6. The method according to claim 5, wherein the carrier includes an excipient, a diluent, a filler, a binder, a wetting agent, a disintegrant, an absorption enhancer, a surfactant, an adsorption carrier, and a stabilizer.

7. The method according to claim 1, wherein the TACI-Fc fusion protein is in a dosage form of an oral preparation or an injection preparation.

8. The method according to claim 4, wherein the TACI-Fc fusion protein is administered in a pharmaceutically acceptable carrier.

9. The method according to claim 8, wherein the carrier comprises an excipient, a diluent, a filler, a binder, a wetting agent, a disintegrant, an absorption enhancer, a surfactant, an adsorption and a stabilizer.

10. The method according to claim 9, wherein the TACI-Fc fusion protein is in a dosage form of an oral preparation or an injection preparation.
